(19) United States
(12) Patent Application Publication
(10) Pub. No.: US 2007/0173890 A1
(43) Pub. Date: Jul. 26, 2007

(54) Stimulation mode adjustment for an implantable medical device

(75) Inventor: Randolph K. Armstrong, Houston, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC.

(21) Appl. No.: 11/338,374

(22) Filed: Jan. 24, 2006

(51) Int. Cl. A61N 1/36

(52) U.S. Cl. 607/2

(57) ABSTRACT

A method and apparatus for providing for altering a neurostimulation therapy provided by an implantable medical device (IMD). A presence of a magnetic field and/or a tap input is detected. A programmed time period for altering the neurostimulation therapy is determined based upon detecting the presence of the magnetic field and/or the tap input. An alteration of the neurostimulation therapy is performed for the duration of the time period.

### CROSS-REFERENCE TO RELATED APPLICATION

This application is a related application to United States patent application Ser. No. ______, entitled “Input Response Override For An Implantable Medical Device,” which is filed on the same date as the present application and in the name of the same inventor.

### BACKGROUND OF THE INVENTION

1. Field of the Invention

This invention relates generally to implantable medical devices, and, more particularly, to methods, apparatus, and systems for providing an alternative stimulation mode for an implantable medical device.

2. Description of the Related Art

There have been many improvements over the last several decades in medical treatments for disorders of the nervous system, such as epilepsy and other motor disorders, and abnormal neural discharge disorders. One of the more recently available treatments involves the application of an electrical signal to reduce various symptoms or effects caused by such neural disorders. For example, electrical signals have been successfully applied at strategic locations in the human body to provide various benefits, including reducing occurrences of seizures and/or improving or ameliorating other conditions. A particular example of such a treatment regimen involves applying an electrical signal to the vagus nerve of the human body to reduce or eliminate epileptic seizures, as described in U.S. Pat. Nos. 4,702,254, 4,867,164, and 5,025,807 to Dr. Jacob Zabara, which are hereby incorporated in their entirety herein by reference in this specification.

More generally, the endogenous electrical activity (i.e., activity attributable to the natural functioning of the patient's own body) of a neural structure of a patient may be modulated in a variety of ways. In particular, the electrical activity may be modulated by exogenously applied (i.e., from a source other than the patient's own body) electrical, chemical, or mechanical signals applied to the neural structure. The modulation (hereinafter referred to generally as “neurostimulation” or “neuromodulation”) may involve the induction of afferent action potentials, efferent action potentials, or both, in the neural structure, and may also involve blocking or interrupting the transmission of endogenous electrical activity traveling along the nerve. Electrical neurostimulation or modulation of a neural structure refers to the application of an exogenous electrical signal (as opposed to a chemical or mechanical signal), to the neural structure. Electrical neurostimulation may be provided by implanting an electrical device underneath the skin of a patient and delivering an electrical signal to a nerve such as a cranial nerve. The electrical neurostimulation may involve performing a detection, with the electrical signal being delivered in response to a detected body parameter. This type of stimulation is generally referred to as “active,” “feedback,” or “triggered” stimulation. Alternatively, the system may operate without a detection system once the patient has been diagnosed with epilepsy (or another medical condition), and may periodically apply a series of electrical pulses to the nerve (e.g., a cranial nerve such as a vagus nerve) intermittently throughout the day, or over another predetermined time interval. This type of stimulation is generally referred to as “passive,” “non-feedback,” or “prophylactic,” stimulation. The stimulation may be applied by an implantable medical device that is implanted within the patient's body, or by a device that is external to the patient's body, with a radio frequency (RF) coupling to an implanted electrode.

Generally, implantable medical devices (IMD) are capable of receiving a signal that may affect the operation of the IMD, from sources external to the IMD, such as a patient-initiated signal or a signal in the patient's environment. For example, a magnetic sensor may be provided in the IMD to detect a significant magnetic field, and in response, activate a predetermined function. A magnetic signal input from a patient may include an inhibitory input or an excitatory input. The inhibitory input may relate to inhibiting a function normally performed by the IMD. For example, application of a particular magnetic field to the IMD may cause delivery of the electrical signal from the IMD to the nerve to be inhibited for a certain time period. Application of a different magnetic field signal to the IMD may prompt the IMD to perform additional functions. For example, based upon a particular magnetic signal input, the IMD may deliver additional stimulation therapy. A patient may generate the magnetic signal input by placing a magnet proximate the skin area under which the implantable medical device resides in the body. Both types of magnetic field signals are typically referred to as “magnet modes” or as “magnet mode” operation.

One problem associated with current magnet mode approaches includes the fact that at times, it may be desirable to suspend normal neurostimulation therapy for prolonged time periods. At other times, it may be desirable to increase the amount of neurostimulation therapy delivered by the IMD using a magnetic signal input. The magnetic signal input may include affixing or taping a magnet upon a skin region under which the IMD resides. Based upon the magnetic signal input, an inhibition of stimulation may be triggered to temporarily reduce various side effects of the neurostimulation therapy, such as hoarseness in the patient's voice.

The state-of-the-art generally lacks an efficient method of inhibiting or altering the operation of the IMD without providing relatively cumbersome solutions, such as taping a magnet on a patient's body or clothing. Additionally, any movement of the magnet relative to the device may cause a false or interrupted input, which may result in the triggering of unsolicited or undesirable neurostimulation therapy, or in a lack of desired neurostimulation therapy. For example, if a patient desires that no neurostimulation take place during a planned speech, a magnet may be taped onto the patient's body or clothing adjacent to IMD's location under the skin to ensure that neurostimulation will be not delivered during the speech, thereby avoiding voice modulation, hoarseness or other vocal problems associated with the neurostimulation. The manual approach may not be convenient or reliable for controlling the operation of the IMD. If the magnet is inadvertently moved or not placed properly, the effect upon the IMD may be sporadic or entirely ineffective.

The manual inhibition process may be inconvenient and may lack the desired reliability. Further, simply affixing the magnet adjacent to the device may not offer sufficient options to regulate the operation of the IMD. For example, a signal to implement a reduced stimulation mode may be indistinguishable from a signal to implement a complete inhibition using the current configurations of IMDs. Additionally, a person entering an area of magnetic activity or fluctuations may cause an IMD to experience false inputs. Current IMD configurations generally lack an effective method of overriding such false inputs.

The present invention is directed to overcoming, or at least reducing, the effects of one or more of the problems set forth above.

### SUMMARY OF THE INVENTION

In one aspect, the present invention comprises a method for altering a neurostimulation therapy provided by an implantable medical device (IMD). An IMD capable of providing a neurostimulation therapy comprising at least a first electrical signal is provided. The IMD is programmed with a time period for altering the neurostimulation therapy. A presence of a magnetic field and/or a tap input is detected. The neurostimulation therapy is altered for the duration of the programmed time period. The alteration comprises at least one of inhibiting the first electrical signal and performing a background stimulation.

In another aspect, the present invention comprises a method for altering a neurostimulation therapy provided by an implantable medical device (IMD). A time period for altering the neurostimulation therapy is programmably defined. An input from a source external to the IMD is received. The neurostimulation therapy is inhibited for the programmably defined time period in response to receiving the input.

In another aspect, the present invention comprises a method for altering a neurostimulation therapy provided by an implantable medical device (IMD). An IMD capable of providing a neurostimulation therapy comprising at least a first electrical signal is provided. The method further comprises determining at least one programmed alteration time period exceeding 60 seconds. The presence of at least one of a magnetic field and a tap input is detected. The neurostimulation therapy is altered for the programmed alteration time period. The alteration comprises at least one of inhibiting the first electrical signal, performing a background stimulation, performing a reduced stimulation, performing a sub-side effect stimulation, and performing an imperceptible stimulation.

### BRIEF DESCRIPTION OF THE DRAWINGS

The invention may be understood by reference to the following description taken in conjunction with the accompanying drawings, in which like reference numerals identify like elements, and in which:

FIGS. 1A-1D provide stylized diagrams of an implantable medical device implanted into a patient's body for providing an electrical signal to a portion of the patient's body, in accordance with one illustrative embodiment of the present invention;

FIG. 2 illustrates a block diagram depiction of the implantable medical device of FIG. 1, in accordance with one illustrative embodiment of the present invention;

FIG. 3 illustrates a more detailed block diagram depiction of a stimulation override unit of FIG. 2, in accordance with one illustrative embodiment of the present invention;

FIG. 4 illustrates a flowchart depiction of a method for performing a stimulation override process, in accordance with a first illustrative embodiment of the present invention;

FIG. 5 illustrates a flowchart depiction of the steps for writing to an override register in relation to the stimulation override process of FIG. 4, in accordance with one illustrative embodiment of the present invention;

FIG. 6 illustrates a flowchart depiction of the steps for monitoring an override register relating to the stimulation override process of FIG. 4, in accordance with one illustrative embodiment of the present invention;

FIG. 7 illustrates a block diagram depiction of the implantable medical device of FIG. 1, in accordance with an alternative illustrative embodiment of the present invention;

FIG. 8 illustrates a more detailed block diagram depiction of a variable stimulation-inhibition unit of FIG. 7, in accordance with one illustrative embodiment of the present invention;

FIG. 9 illustrates a flowchart depiction of a method of implementing a variable stimulation process, in accordance with a second illustrative embodiment of the present invention; and

FIG. 10 illustrates a flowchart depiction of the steps for providing the timing for the variable stimulation process of FIG. 9, in accordance with one illustrative embodiment of the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described herein. In the interest of clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

Embodiments of the present invention provide for an input to the IMD that would prompt the IMD to operate in an alternative mode for a predetermined period of time, or until another triggering input is received. Embodiments of the present invention provides for flexibility in controlling the operation of the IMD.

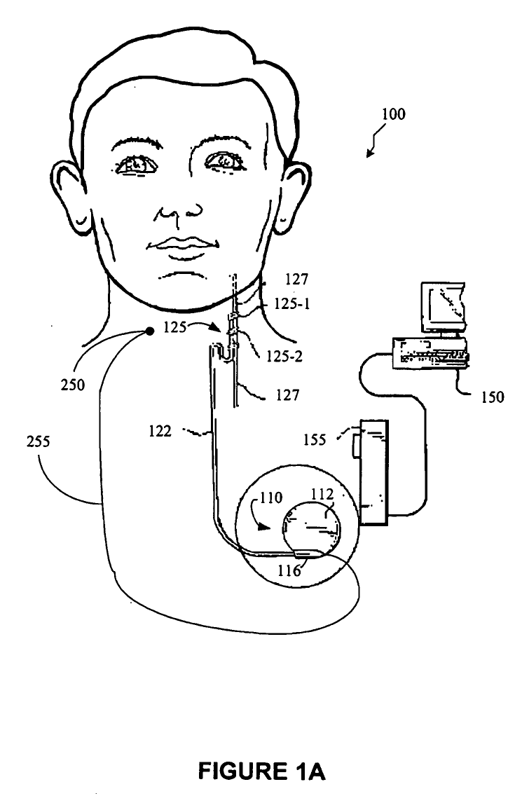

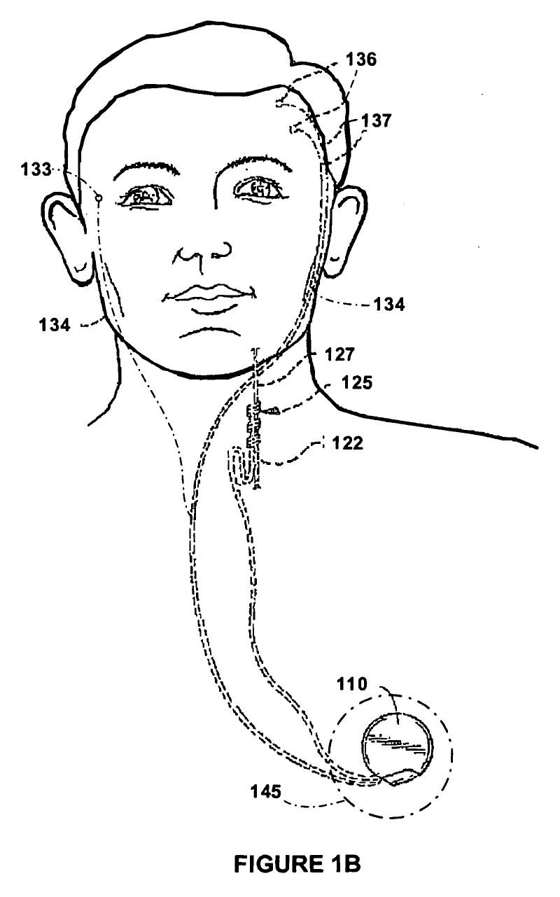

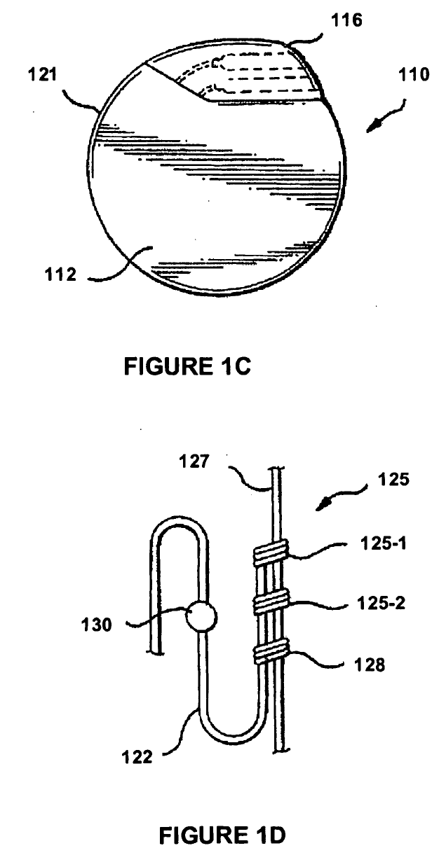

Although not so limited, a system capable of implementing embodiments of the present invention is described below. FIGS. 1A-1D depict a stylized implantable medical system 100 for implementing one or more embodiments of the present invention. FIGS. 1A-1D illustrate an electrical signal generator 110 having main body 112 comprising a case or shell 121 (FIG. 1A) with a header 116 (FIG. 1C) for connecting to leads 122. The generator 110 is implanted in the patient's chest in a pocket or cavity formed by the implanting surgeon just below the skin (indicated by a dotted line 145, FIG. 1B), similar to the implantation procedure for a pacemaker pulse generator.

A stimulating nerve electrode assembly 125, preferably comprising an electrode pair, is conductively connected to the distal end of an insulated, electrically conductive lead assembly 122, which preferably comprises a pair of lead wires (one wire for each electrode of an electrode pair). Lead assembly 122 is attached at its proximal end to connectors on the header 116 (FIG. 1C) on case 121. The electrode assembly 125 may be surgically coupled to a vagus nerve 127 in the patient's neck or at another location, e.g., near the patient's diaphragm. Other cranial nerves may also be used to deliver the electrical neurostimulation signal. The electrode assembly 125 preferably comprises a bipolar stimulating electrode pair 125-1, 125-2 (FIG. 1D), such as the electrode pair described in U.S. Pat. No. 4,573,481 issued Mar. 4, 1986 to Bullara. Suitable electrode assemblies are available from Cyberonics, Inc., Houston, Tex., USA as the Model 302 electrode assembly. However, persons of skill in the art will appreciate that many electrode designs could be used in the present invention. The two electrodes are preferably wrapped about the vagus nerve, and the electrode assembly 125 may be secured to the nerve 127 by a spiral anchoring tether 128 (FIG. 1D) such as that disclosed in U.S. Pat. No. 4,979,511 issued Dec. 25, 1990 to Reese S. Terry, Jr. and assigned to the same assignee as the instant application. Lead assembly 122 is secured, while retaining the ability to flex with movement of the chest and neck, by a suture connection 130 to nearby tissue (FIG. 1D).

In one embodiment, the open helical design of the electrode assembly 125 (described in detail in the above-cited Bullara patent), which is self-sizing and flexible, minimizes mechanical trauma to the nerve and allows body fluid interchange with the nerve. The electrode assembly 125 preferably conforms to the shape of the nerve, providing a low stimulation threshold by allowing a large stimulation contact area with the nerve. Structurally, the electrode assembly 125 comprises two electrode ribbons (not shown), of a conductive material such as platinum, iridium, platinum-iridium alloys, and/or oxides of the foregoing. The electrode ribbons are individually bonded to an inside surface of an elastomeric body portion of the two spiral electrodes 125-1 and 125-2 (FIG. 1D), which may comprise two spiral loops of a three-loop helical assembly. The lead assembly 122 may comprise two distinct lead wires or a coaxial cable whose two conductive elements are respectively coupled to one of the conductive electrode ribbons. One suitable method of coupling the lead wires or cable to the electrodes 125-1, 125-2 comprises a spacer assembly such as that disclosed in U.S. Pat. No. 5,531,778, although other known coupling techniques may be used.

The elastomeric body portion of each loop is preferably composed of silicone rubber, and the third loop 128 (which typically has no electrode) acts as the anchoring tether for the electrode assembly 125.

In certain embodiments of the invention, sensors such as eye movement sensing electrodes 133 (FIG. 1B) may be implanted at or near an outer periphery of each eye socket in a suitable location to sense muscle movement or actual eye movement. The electrodes 133 may be electrically connected to leads 134 implanted via a catheter or other suitable means (not shown) and extending along the jaw line through the neck and chest tissue to the header 116 of the electrical pulse generator 110. When included in systems of the present invention, the sensing electrodes 133 may be utilized for detecting rapid eye movement (REM) in a pattern indicative of a disorder to be treated, as described in greater detail below. The detected indication of the disorder can be used to trigger active stimulation.

Other sensor arrangements may alternatively or additionally be employed to trigger active stimulation. Referring again to FIG. 1B, electroencephalograph (EEG) sensing electrodes 136 may optionally be implanted and placed in spaced-apart relation on the skull, and connected to leads 137 implanted and extending along the scalp and temple, and then connected to the electrical pulse generator 110 along the same path and in the same manner as described above for the eye movement electrode leads 134.

In alternative embodiments, temperature sensing elements and/or heart rate sensor elements may be employed to trigger active stimulation. In addition to active stimulation incorporating sensor elements, other embodiments of the present invention utilize passive stimulation to deliver a continuous, periodic or intermittent electrical signal (each of which constitutes a form of continual application of the signal) to the vagus nerve according to a programmed on/off duty cycle without the use of sensors to trigger therapy delivery. Both passive and active stimulation may be combined or delivered by a single IMD according to the present invention. Either or both modes may be appropriate to treat the particular disorder diagnosed in the case of a specific patient under observation.

The electrical pulse generator 110 may be programmed with an external computer 150 using programming software of the type copyrighted by the assignee of the instant application with the Register of Copyrights, Library of Congress, or other suitable software based on the description herein, and a programming wand 155 to facilitate radio frequency (RF) communication between the computer 150 (FIG. 1A) and the pulse generator 110. The wand 155 and software permit non-invasive communication with the generator 110 after the latter is implanted. The wand 155 is preferably powered by internal batteries, and provided with a “power on” light to indicate sufficient power for communication. Another indicator light may be provided to show that data transmission is occurring between the wand and the generator.

A variety of stimulation therapies may be provided in implantable medical systems 100 of the present invention. Different types of nerve fibers (e.g., A, B, and C fibers being different fibers targeted for stimulation) respond differently to stimulation from electrical signals. More specifically, the different types of nerve fibers have different conduction velocities and stimulation thresholds and, therefore, differ in their responsiveness to stimulation. Certain pulses of an electrical stimulation signal, for example, may be below the stimulation threshold for a particular fiber and, therefore, may generate no action potential in the fiber. Thus, smaller or narrower pulses may be used to avoid stimulation of certain nerve fibers (such as C fibers) and target other nerve fibers (such as A and/or B fibers, which generally have lower stimulation thresholds and higher conduction velocities than C fibers). Additionally, techniques such as pre-polarization may be employed wherein particular nerve regions may be polarized before a more robust stimulation is delivered, which may better accommodate particular electrode materials. Furthermore, opposing polarity phases separated by a zero current phase may be used to excite particular axons or postpone nerve fatigue during long-term stimulation.

As used herein, the terms “stimulating” and “stimulator” may generally refer to delivery of a signal, stimulus, or impulse to neural tissue for affecting neuronal activity of a neural tissue (e.g., a volume of neural tissue in the brain or a nerve). The effect of such stimulation on neuronal activity is termed “modulation”; however, for simplicity, the terms “stimulating” and “modulating”, and variants thereof, are sometimes used interchangeably herein. The effect of delivery of the stimulation signal to the neural tissue may be excitatory or inhibitory and may potentiate acute and/or long-term changes in neuronal activity. For example, the effect of “stimulating” or “modulating” a neural tissue may comprise on one more of the following effects: (a) changes in neural tissue to initiate an action potential (bi-directional or uni-directional); (b) inhibition of conduction of action potentials (endogenous or externally stimulated) or blocking the conduction of action potentials (hyperpolarizing or collision blocking), (c) affecting changes in neurotransmitter/neuromodulator release or uptake, and (d) changes in neuro-plasticity or neurogenesis of brain tissue. Applying an electrical signal to an autonomic nerve may comprise generating a response that includes an afferent action potential, an efferent action potential, an afferent hyperpolarization, an efferent hyperpolarization, an afferent sub-threshold depolarization, and/or an efferent sub-threshold depolarization.

Embodiments of the present invention provide for performing an override of one or more safety features based upon one or more external inputs received by the IMD. For example, the IMD may receive various inputs that could prompt a temporary interruption or deviation from normal stimulation operation. For example, a magnet may be placed proximate to the IMD, which may be an indication that the patient or a physician desires to alter the normal operation of the MD. The amount of time that the magnet is detected may determine the type of deviation from the normal operation that will occur. Various devices, such as a Reed Switch or a Hall Effect sensor may be employed to detect a magnetic field in order to react to a magnet being placed proximate to the MD.

Embodiments of the present invention provide for overriding the presence of a magnetic field using various techniques. For example, software techniques may be used to override the presence of a reaction to the presence of a magnetic field based on an earlier input or another indication provided to the IMD. Other techniques, such as hardware, firmware circuits, etc., may be used to monitor a register to determine whether to ignore the interruption data deciphered by a magnetic sensor. This may be beneficial when the patient enters a magnetic field area, such as an MRI field or other electromagnetic location(s).

Further, an external input received by the IMD may be used to temporarily alter the normal operation of the IMD. For example, the patient may desire to temporarily stop all stimulation activity. Alternatively, an input from the patient (e.g., a magnetic input) may be used to provide a second electrical signal for a programmed time period. Providing the second electrical signal may be accompanied by inhibiting the first electrical signal. The second electrical signal may comprise a background signal, a “reduced” or “sub-side effect” signal that reduces or eliminates certain stimulation side effects, or both. The amount of time to employ the alternative stimulation mode, as well as the type of alternative stimulation mode, may be programmed into the IMD.

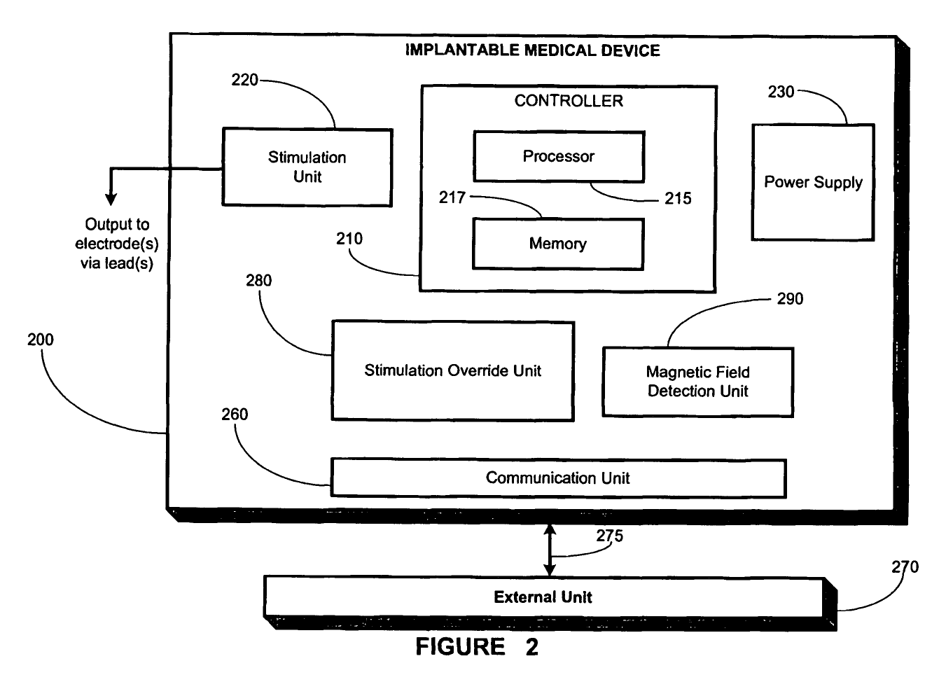

Turning now to FIG. 2, a block diagram depiction of an implantable medical device, in accordance with one illustrative embodiment of the present invention is illustrated. The IMD 200 may be used for stimulation to treat various disorders, such as epilepsy, depression, bulimia, heart rhythm disorders, gastric-related disorder, a hormonal disorder, a reproductive disorder, a metabolic disorder, a hearing disorder, and/or a pain disorder. The IMD 200 may be coupled to various leads, e.g., 122, 134, 137 (FIGS. 1A, 1B, ID). Stimulation signals used for therapy may be transmitted from the IMD 200 to target areas of the patient's body, specifically to various electrodes associated with the leads 122. Stimulation signals from the IMD 200 may be transmitted via the leads 122 to stimulation electrodes associated with the electrode assembly 125 (FIG. 1A). Further, signals from sensor electrodes, e.g., 133, 136 (FIG. 1B) associated with corresponding leads, e.g., 134, 137, may also traverse the leads back to the IMD 200.

The IMD 200 may comprise a controller 210 capable of controlling various aspects of the operation of the IMD 200. The controller 210 is capable of receiving internal data and/or external data and generating and delivering a stimulation signal to target tissues of the patient's body. For example, the controller 210 may receive manual instructions from an operator externally, or may perform stimulation based on internal calculations and programming. The controller 210 is capable of affecting substantially all functions of the IMD 200.

The controller 210 may comprise various components, such as a processor 215, a memory 217, etc. The processor 215 may comprise one or more micro controllers, microprocessors, etc., that are capable of executing a variety of software components. The memory 217 may comprise various memory portions, where a number of types of data (e.g., internal data, external data instructions, software codes, status data, diagnostic data, etc.) may be stored. The memory 217 may store various tables or other database content that could be used by the IMD 200 to implement the override of normal operations. The memory 217 may comprise random access memory (RAM) dynamic random access memory (DRAM), electrically erasable programmable read-only memory (EEPROM), flash memory, etc.

The IMD 200 may also comprise a stimulation unit 220. The stimulation unit 220 is capable of generating and delivering a variety of electrical neurostimulation signals to one or more electrodes via leads. The stimulation unit 220 is capable of generating a therapy portion, a ramping-up portion, and a ramping-down portion of the stimulation signal. A number of leads 122, 134, 137 may be coupled to the IMD 200. Therapy may be delivered to the leads 122 by the stimulation unit 220 based upon instructions from the controller 210. The stimulation unit 220 may comprise various types of circuitry, such as stimulation signal generators, impedance control circuitry to control the impedance “seen” by the leads, and other circuitry that receives instructions relating to the type of stimulation to be performed. The stimulation unit 220 is capable of delivering a controlled current stimulation signal to the leads and to the electrodes the leads 122.

The IMD 200 may also comprise a power supply 230. The power supply 230 may comprise a battery, voltage regulators, capacitors, etc., to provide power for the operation of the IMD 200, including delivering the stimulation signal. The power supply 230 comprises a power-source battery that in some embodiments may be rechargeable. In other embodiments, a non-rechargeable battery may be used. The power supply 230 provides power for the operation of the IMD 200, including electronic operations and the stimulation function. The power supply 230 may comprise a lithium/thionyl chloride cell or a lithium/carbon monofluoride cell. Other battery types known in the art of implantable medical devices may also be used.

The IMD 200 also comprises a communication unit 260 capable of facilitating communications between the IMD 200 and various devices. In particular, the communication unit 260 is capable of providing transmission and reception of electronic signals to and from an external unit 270. The external unit 270 may be a device that is capable of programming various modules and stimulation parameters of the IMD 200. In one embodiment, the external unit 270 comprises a computer system that is capable of executing a data-acquisition program. The external unit 270 may be controlled by a healthcare provider, such as a physician, at a base station in, for example, a doctor's office. The external unit 270 may be a computer, preferably a handheld computer or PDA, but may alternatively comprise any other device that is capable of electronic communications and programming. The external unit 270 may download various parameters and program software into the IMD 200 for programming the operation of the implantable device. The external unit 270 may also receive and upload various status conditions and other data from the IMD 200. The communication unit 260 may be hardware, software, firmware, and/or any combination thereof. Communications between the external unit 270 and the communication unit 260 may occur via a wireless or other type of communication, illustrated generally by line 275 in FIG. 2.

The IMD 200 is capable of delivering stimulation that can be intermittent, periodic, random, sequential, coded, and/or patterned. The stimulation signals may comprise an electrical stimulation frequency of approximately 0.1 to 2500 Hz. The stimulation signals may comprise a pulse width of in the range of approximately 1-2000 microseconds. The stimulation signals may comprise current amplitude in the range of approximately 0.1 mA to 10 mA. Stimulation may be delivered through either the cathode (−) electrode or anode (+) electrode. In one embodiment, the various blocks illustrated in FIG. 2 may comprise software unit, a firmware unit, a hardware unit, and/or any combination thereof.

The IMD 200 may also comprise a magnetic field detection unit 290. The magnetic field detection unit 290 is capable of detecting magnetic and/or electromagnetic fields of a predetermined magnitude. Whether the magnetic field results from a magnet placed proximate to the IMD 200, or whether it results from a substantial magnetic field encompassing an area, the magnetic field detection unit 290 is capable of informing the IMD of the existence of a magnetic field.

The magnetic field detection unit 270 may comprise various sensors, such as a Reed Switch circuitry, a Hall Effect sensor circuitry, and/or the like. The magnetic field detection unit 270 may also comprise various registers and/or data transceiver circuits that are capable of sending signals that are indicative of various magnetic fields, the time period of such fields, etc. In this manner, the magnetic field detection unit 270 is capable of deciphering whether the detected magnetic field relates to an inhibitory input or an excitory input from an external source. The inhibitory input may refer to an inhibition of, or a deviation from, normal stimulation operation. The excitory input may refer to additional stimulation or deviation from normal stimulation.

The IMD 200 may also include a stimulation override unit 280. The stimulation override unit 280 is capable of overriding the reaction by the IMD to the detection of a magnetic signal provided by the magnetic field detection unit 270. The stimulation override unit 280 may comprise various software, hardware, and/or firmware units that are capable of determining an amount of time period in which to override the detection of a magnetic field. The stimulation override unit 280 may also contain safety features, such as returning to normal operation despite an override command after a predetermined period of time. The stimulation override unit 280 is capable of preventing false interruption of normal operation due to false magnetic input signals or unintended magnetic input signals. The stimulation override unit 280 may receive an external indication via the communication unit 270 to engage in an override mode for a predetermined period of time.

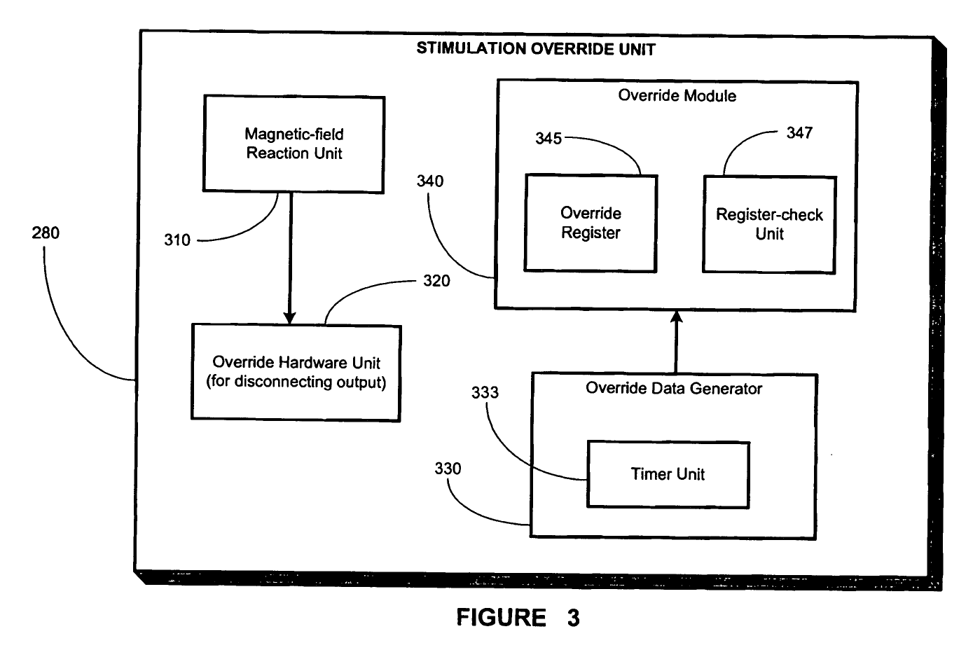

Turning now to FIG. 3, a more detailed block diagram depiction of the stimulation override unit 280 of FIG. 2, is illustrated. In one embodiment, the stimulation override unit 280 comprises a magnetic field reaction unit 310. The magnetic-field reaction unit 310 may determine how to react to a magnetic field detected by the magnetic-field detection unit 270 (FIG. 2). The magnetic field reaction unit 310 may provide a signal to the IMD 200 to either stop stimulation or to alter the stimulation in some fashion.

The stimulation override unit 280 may also comprise an override hardware unit 320. Based upon data from the magnetic field reaction unit 310, the override hardware unit 320 may disconnect the stimulation signal from the leads or electrodes that may be coupled to the IMD 200. The override hardware unit 320 may comprise various devices, such as switches, registers, multiplexers, etc., that are capable of receiving data and disconnecting stimulation signals to various output ports of the IMD 200, which may be coupled to leads or electrodes.

The stimulation override unit 280 may also comprise an override module 340. The override module 340 is capable of monitoring a predetermined data location to determine whether to continue with an override of a reaction to a magnetic signal. The override module 340 may comprise an override register 345 and a register-check unit 347. The register-check unit 347 is capable of monitoring data in the override register 345. In order to maintain an override mode, data may be written to the override register 345 in a periodic predetermined fashion. The register-check unit 347 then monitors the override register 345 at a predetermined frequency. When the override check unit 347 determines that the override register 345 contains the appropriate override data, the override module 340 maintains the override mode of the IMD 200. When the register-check unit 347 determines that the appropriate override data does not exist in the override register 345, the register-check unit 347 may then prompt the override module to exit the override mode and enter into a normal stimulation mode.

The override register 345 may comprise circuitry that, by default, may register “fill” data, e.g., a predetermined string of 0's, 1's, or any combination thereof. (e.g., six consecutive 0's followed by three two 1's). Therefore, an affirmative registering of override data being periodically written into the override register 345 may be required for the override module 340 to maintain the override mode. Therefore, without active, intentional action by the IMD 200 to maintain the override mode, the default may be to fall back to normal stimulation mode.

The stimulation override unit 280 may also comprise an override data generator 330. The override data generator 330 may generate the override data that is registered into the override register 345 in the override module 340. The override data may comprise a predetermined string of data with a specific pattern (e.g., six consecutive 1's followed by two 0's). The override data generator 330 may receive data from the communication unit 260 to prompt the generation of the override mode.

The override data generator 330 may also receive data relating to the time period in which the IMD 200 is to be in an override mode. The override register data generator 330 may comprise a timer unit 333, which is capable of controlling the time period in which the override mode is to be active. Upon indication from the timer unit 333 that the override mode time period has expired, the override data generator 330 stops sending data to the override register 345. Based upon this action, the override register 345 may then be filled with default fill data, such as a stream of 0's. This would prompt the override module 340 to exit the override mode and prompt the IMD 200 to enter a normal operation mode.

Various blocks illustrated in FIG. 3 may be individual modules, such as software modules (e.g., object-oriented code, subroutines, etc.), hardware modules, and/or firmware modules (e.g., programmable gate arrays, ASIC-related modules, hardware description language (HDL) modules, etc.). Alternatively, two or more blocks in FIG. 3 may be merged together into one or more software modules, hardware modules, and/or firmware modules.

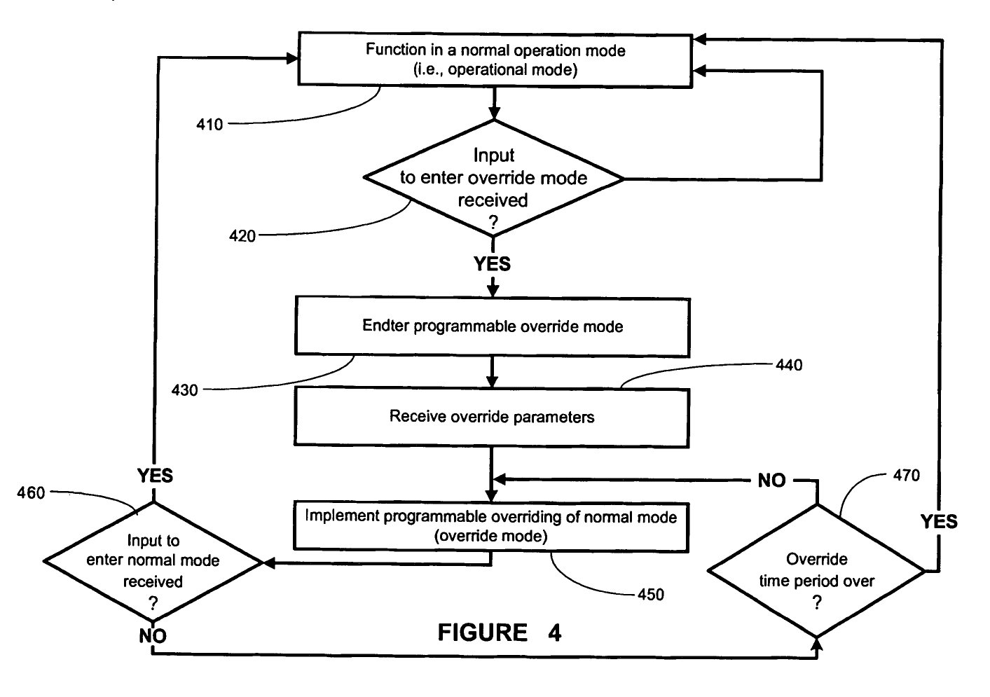

Turning now to FIG. 4, a flowchart depiction of the method for performing the override mode in accordance with one illustrative embodiment of the present invention is provided. Initially, the IMD 200 may be operating in a normal operation mode (block 410). The normal operation mode calls for predetermined delivery of stimulation signals followed by inactive or diminished active time periods that are interspersed between actual stimulation cycles. The IMD 200 may then check to determine whether an input to enter an override mode has been received (block 420). If an input to enter an override mode has not been received, normal operation of the IMD and delivery of stimulation signal is resumed, as indicated in FIG. 4. However, if it is determined that an input signal prompting an entry into an override mode has been detected, the IMD 200 may enter a programmable override mode (block 430).

The programmable override mode may refer to a predetermined override mode that may be programmed into the IMD 200 by the patient or a physician. Various inputs to enter the override mode may be provided, such as a magnetic input, a tap input, wireless data transfer via the communication line 375, etc. The IMD 200 may then receive or lookup the relevant override parameters (block 440). Various override parameters may be received, such as the time period for the override, the type of override, e.g., whether a complete shut down of stimulation is required, or whether a modification of the type of stimulation is required.

Upon receiving the override parameters, the IMD 200 implements the programmable override mode. This includes activating the stimulation override unit 280 to cause the IMD 200 to enter into an alternative operation mode (block 450). A determination may then be made whether an input has been received prompting the IMD 200 to go back to a normal mode of operation (block 460). When a determination is made that the normal operation input has not been received, the override programmable mode is continued. Upon a determination that the input to resume normal operation is received, the IMD 200 resumes normal operations. Additionally, upon implementation of the programmable override, a check is made to determine whether the time period for the override mode has expired (block 470). If the time period for the override mode has not expired, the override programmable override mode is continued. However, when the time period for override mode has expired, normal operation is then resumed, as indicated by the path from block 470 to block 410. In this manner, the override function may be programmable and predetermined, wherein a patient entering a magnetic-field area may program the IMD 200 to not perform overriding activities for a predetermined period of time.

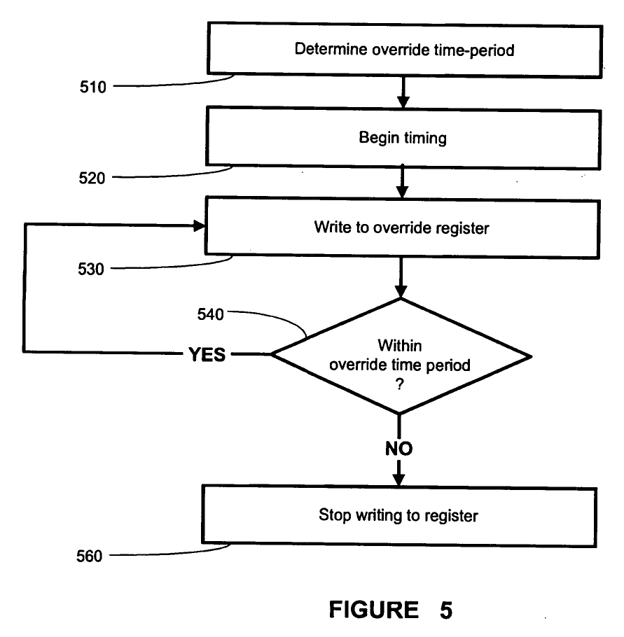

Turning now to FIG. 5, a flowchart depiction relating to the timing of performing the override mode implementation of FIG. 4, in accordance with one illustrative embodiment of the present invention, is provided. The IMD 200 may determine the override time period (block 510). The override time period may be pre-programmed into the IMD 200 or may be received as an external input. Upon determining the time period for the override mode, the timer unit 333 and the override data generator 330 (FIG. 3) may perform a timing function (block 520).

Upon beginning the timing function, the override data generator 330 may write data into the override register (block 530). The data that is written to the override register may include predetermined override data, which may be indicative of the type of override to perform. This data may be indicative of various types of override that may be performed, such as complete elimination of stimulation, modification of the stimulation cycle pulse width, amplitude, and the like. Upon writing to the override register 345, a check may be made to determine whether the time period to perform the override mode has expired (block 540). When it is determined that the time period for the override mode has not expired, override data is periodically written into the override register 345 to maintain the override mode (block 550). Upon a determination that the time period to perform the override mode has expired, the override register generator 330 stops writing data into the override register (block 560). This would cause default data to be registered into the override register 345, thereby causing the override module 340 to stop the override mode and enter into a normal stimulation mode.

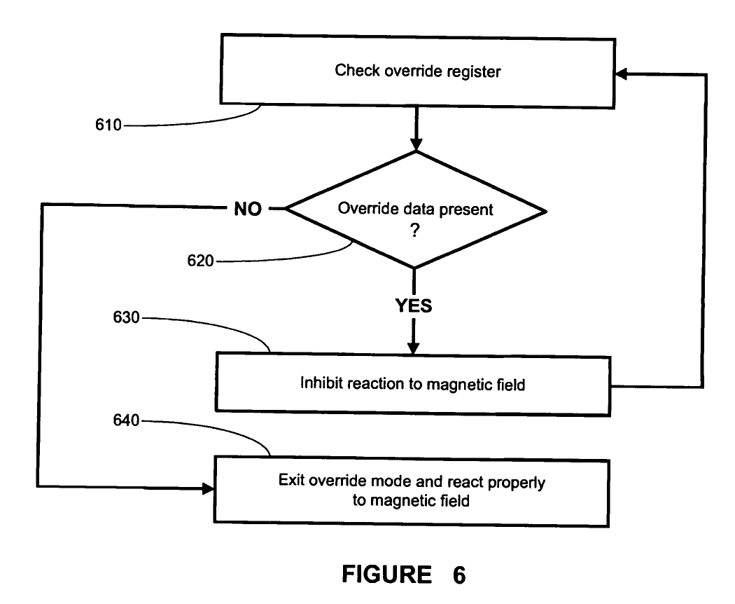

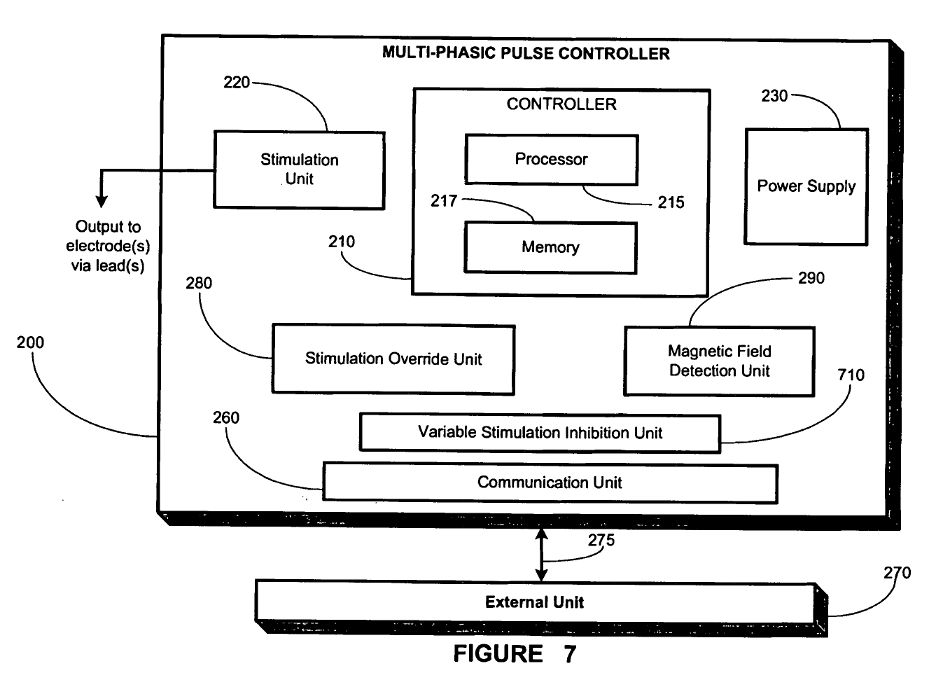

Turning now to FIG. 6, a flowchart depiction of the step of determining whether to to maintain an override mode, is illustrated. The override module 340 may check the override register 345 to determine what type of data is found (block 610). The override module 340 determines whether override data is present in the override register 345 (block 620). If it is determined that the override data is indeed present in the override register 345, the IMD 200 inhibits the reaction to the magnetic field (block 630). In other words, the IMD 200 continues with normal operation and prevents the normal default safety-stoppage that would have occurred but for the data present in the override register 345.

The override module 340 then continues to check the override register at a predetermined frequency and repeats the process described in block 610, 620 and 630 of FIG. 6. Upon a determination that the override data present is not present in the override register, the IMD 200 may exit the override mode and return to normal reaction to the magnetic field (block 640). In other words, the IMD returns to the inhibition or alteration of the normal stimulation process based upon the detection of the magnetic field. In this manner, the patient or a physician may override the predetermined safety features that would have cut-off normal stimulation, or alter normal stimulation based upon the detection of a magnetic signal. Therefore, a patient may enter an area that contains significant amount of electromagnetic signals without undesired interruption of the normal stimulation operations of the IMD 200.

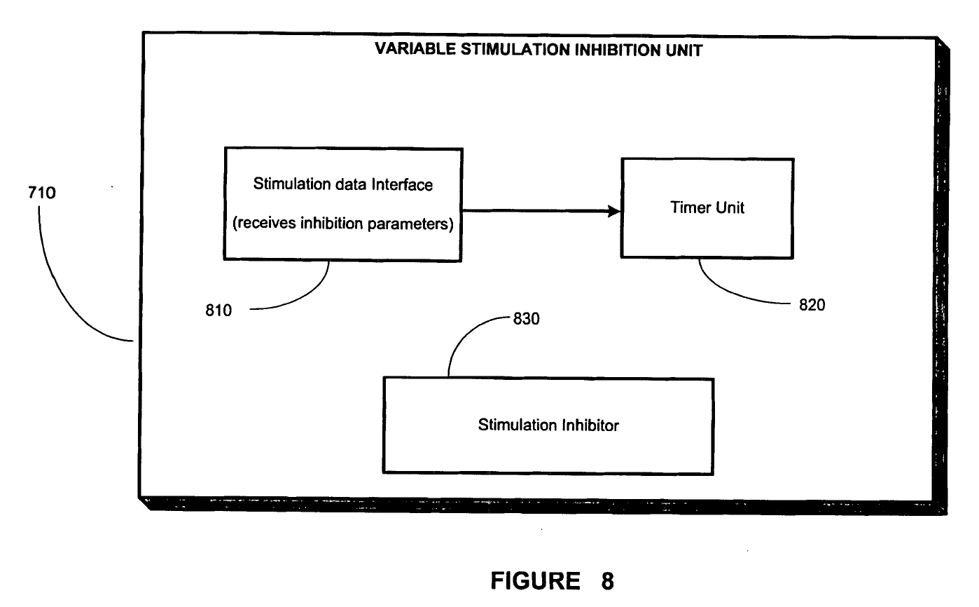

Turning now to FIG. 7, a block diagram depiction of the IMD 200, in accordance with an alternative embodiment of the present invention is illustrated. In addition to the various components described in FIG. 2, and the accompanying descriptions above, the illustrative IMD 200 in FIG. 7 also comprises a variable stimulation-inhibition unit 710. The variable stimulation-inhibition unit 710 is capable of performing a variable inhibition of the normal stimulation operation of the IMD 200. Based upon input received by the IMD 200, such as programmed data received through the communication unit 260 from an external source 270 (e.g., the patient, a physician, etc), the IMD 200 is capable of varying the normal stimulation protocol for a controllable, programmable period of time. The variable stimulation-inhibition unit 710 may comprise various software, hardware, and/or firmware units that are capable of monitoring external data to prompt the IMD 200 to enter into alternative stimulation modes. The alternative stimulation modes may include, but are not limited to, a reduced or sub-side effect stimulation mode, a background stimulation mode, a stimulation mode with modified parameters (e.g., frequency, phase-characteristics, amplitude, polarity, etc), zero stimulation, etc. A more detailed description of the variable stimulation-inhibition unit 710 is provided below in FIG. 8 and accompanying description below.

Turning now to FIG. 8, a more detailed block diagram depiction of the variable stimulation-inhibition unit 710 is illustrated. The variable stimulation-inhibition unit 710 may comprise a stimulation data interface 810. The stimulation data interface 810 is capable of receiving data that may be used to control the type of inhibition or alteration of the normal stimulation process, e.g., a first electrical signal delivering a neurostimulation therapy. The stimulation data interface 810 may receive variable stimulation data from an external source. In this manner, the inhibition or alteration of the normal stimulation process may be pre-programmed in a conventional manner or in a real-time fashion. Various parameters, such as the time period of the inhibition or alteration of normal stimulation, the type of alternative stimulation to be delivered (e.g. reduced stimulation or zero stimulation), etc., may be received by the stimulation data interface 810. Based upon the data received by the stimulation data interface 810, a timer circuit 820 in the variable inhibition unit 710 is capable of controlling the time period in which the alternative stimulation period is implemented.

The variable stimulation-inhibition unit 710 also comprises a stimulation inhibitor (block 830). The stimulation inhibitor 830 may comprise various hardware, software, and/or firmware circuitry that are capable of inhibiting or altering the type of stimulation that is delivered to the patient. Based upon the data provided by the stimulation data interface 810, different types of stimulation may be delivered, such as stimulation with an alternative frequency, amplitude, pulse width, polarity, phases, etc., or a complete termination of any stimulation. Additionally, the stimulation inhibitor 830 is capable of implementing a background stimulation mode during the time period determined by the timer unit 820.

“Background stimulation” refers to a second electrical signal that is delivered during a second time period, wherein a normal stimulation mode is implemented by providing a first electrical signal in a first time period. The second time period for the background stimulation occurs at least partly, and preferably entirely, during an off-time of the first electrical signal. In some embodiments the background stimulation may also comprise a reduced simulation mode. “Reduced stimulation” refers to a second electrical signal in which at least one parameter defining the second signal is less than a corresponding value defining the first electrical signal. One form of reduced stimulation is “imperceptible stimulation”, in which the second electrical signal is provided at a level that is substantially imperceptible to a patient. Another form of reduced stimulation is “sub-side effect stimulation,” which refers to a second electrical signal that provides a reduction or elimination of side effects experienced by the patient, such as voice alteration, as a result of the first electrical signal. Altered stimulation modes may embody a second signal that is simultaneously a background stimulation, a reduced stimulation, an imperceptible stimulation and a sub-side effect stimulation. More generally, an altered stimulation mode may be provided in which a first electrical signal is inhibited and a second electrical signal, which may comprise a background stimulation, a reduced stimulation, an imperceptible stimulation, or a sub-side effect stimulation, may be provided for the programmed time period.

A second electrical signal provided during an off-time of the first signal may thus be referred to hereinafter as “background” stimulation or modulation. For example, an IMD 200 may apply a second electrical signal having a reduced frequency, current, or pulse width relative to the first electrical signal during off-time of the first period, in addition to the first electrical signal applied during a primary period. Without being bound by theory, applying a background electrical signal may allow the first electrical signal to be reduced to a level sufficient to reduce one or more side effects without reducing therapeutic efficacy. Alternatively, the first electrical signal may be completely inhibited during the programmed time period, and a background electrical signal may be applied that itself comprises a reduced signal and/or a sub-side-effect signal.

In some embodiments of the present invention, the first and second time periods at least partially overlap, and a second electrical stimulation signal may be applied during at least a portion of the first time period. In a more particular embodiment, the second time period only partially overlaps the first, and the second electrical stimulation signal is applied during a portion of the first time period and continues during a period in which the first signal is not applied. This type of stimulation is referred to hereinafter as “overlaid” stimulation or modulation. Overlaid and/or background stimulation embodiments of the invention may increase efficacy of a stimulation therapy, reduce side effects, and/or increase tolerability of the first signal to higher levels of stimulation.

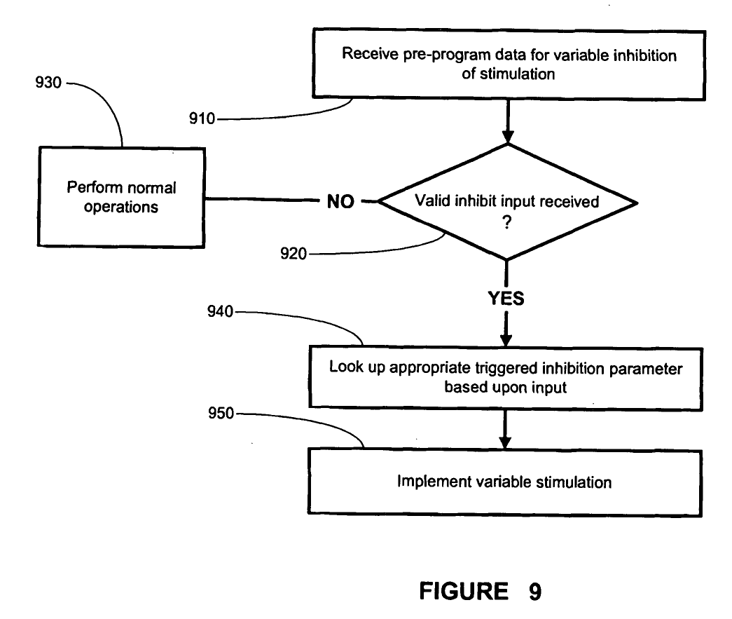

Turning now to FIG. 9, a flowchart depiction of the method of performing the stimulation inhibition mode in accordance with one illustrative embodiment of the present invention is provided. The IMD 200 may receive pre-programmed data for implementing a variable inhibition of the normal stimulation operation (block 910). This pre-preprogrammed data may include the type of alternative stimulation process to be implemented based upon a predetermined input that may trigger the inhibition mode. For example, a tap or a magnetic input provided by the patient may initiate an inhibition stimulation mode where the normal or current stimulation process is altered. As an example, if a person is scheduled to deliver a speech, due to the concern of interference with the person's voice being altered by the delivery of a stimulation signal, normal stimulation operation may be interrupted for a predetermined duration of time. Alternatively, a background stimulation or a zero stimulation may be performed during the predetermined time period. The predetermined time period and the type of alternative stimulation period to enter may be pre-programmed into the IMD 200.

The IMD 200 determines whether the appropriate inhibition input data is received (block 920). If valid inhibition data input is not received, normal stimulation operation is performed (block 930). However, upon a determination that valid stimulation inhibition input is received, such as a tap input or a predetermined magnetic input for a predetermined duration of time, the IMD 200 may look up the appropriate triggered inhibition parameter based upon the input (block 940). In other words, based upon the type of initiation input received, a particular type of inhibition parameter that may be stored in memory may be retrieved. Based upon the inhibition parameter, a preprogrammed implementation of a variable stimulation inhibition mode may be initiated (block 950). This may include examples such as temporarily shutting off any stimulation, entering a background stimulation mode for a predetermined period of time, etc.

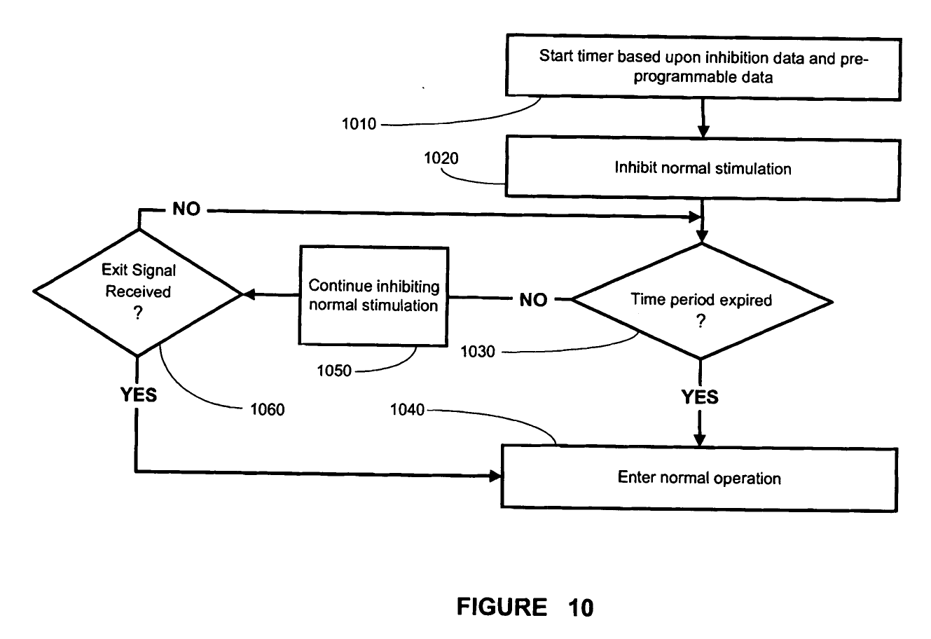

Turning now to FIG. 10, a flowchart depiction of the timing process relating to the stimulation inhibition process is illustrated. The variable stimulation-inhibition unit 710 may initiate the starting of a timer based upon the inhibition data and the preprogrammed data relating to the inhibition mode (block 1010). For example, based upon the type of input received, and the preprogrammed parameters relating to the particular input, the timer may begin measuring a time period for performing a variable stimulation process. Based upon the time period, the IMD 200 performs inhibition of the normal stimulation process, which may provide for preventing any stimulation or entering into an alternative stimulation mode, such as a background stimulation mode (block 1020).

A determination may then be made as to whether the time period for performing the variable stimulation has expired (block 1030). The time period for performing the variable stimulation may be pre-programmed into the IMD 200. For example, the IMD 200 may host an algorithm that directs the IMD 200 to implement an alternative or variable stimulation mode for a time period prescribed by the algorithm. In one embodiment, the pre-programming of the time period may entail an overall time period for alternative stimulation. This overall or maximum time period may be divided into various portions wherein the time period for an alternative stimulation may be a fraction of the overall time period. The time period portion for alternative stimulation may be chosen by the patient or another external input. For example, a particular type of magnetic input or tap signal may indicate a time period for stimulation that is half of the maximum pre-programmed time period. As an example, the maximum time period for alternative stimulation may zero seconds to 64,000 seconds. However, based upon a predetermined input, any sub-division of the 64,000 seconds may be used as the time period for stimulation, after which the time period for performing the variable or alternative stimulation expires.

Increments of the maximum programmed time period for performing alternative stimulation may be selected by various magnetic inputs, which includes duration of a magnetic signal, etc. or a particular type of tap input. For example some selection of the time period for alternative stimulation may be based upon the numbers of taps counted. A one count may, for example, be treated as a probable accident, and ignored. On the other hand, a sequence of two or three taps may be used to select a first division of the time period, e.g., half of the maximum programmed time period. A sequence of four or five taps is used, may be used to select a second division of the maximum programmed time period. Finally, a sequence of seven taps may be used to prompt an exit of the variable stimulation mode. These features that allow the patient to control the operation of the variable stimulation mode may be pre-programmed into the IMD 200.

Based upon an indication that the time period for performing the variable stimulation has expired, the IMD 200 enters into a normal stimulation operation mode (block 1040). Based upon a determination that the time period for the variable stimulation has not expired, the inhibition of the normal stimulation process is continued (block 1050).

A determination may also be made as to whether an external signal to exit the inhibition mode has been received (block 1060). At any time, the patient or the physician may provide a signal to the IMD 200 indicating that the inhibition process is to be terminated and normal stimulation operation is to be resumed. If the signal for exiting the inhibition process has been received, normal stimulation operation is then continued (block 1040). However, if it is determined that the signal for exiting the stimulation process has not been received, the IMD 200 continues to check whether it is within the time period for the inhibition of the normal stimulation process, as illustrated in FIG. 10. In this manner, the alternative stimulation process or the full inhibition of the normal stimulation process is continued until a predetermined time period has expired, or an external input signaling stimulation inhibition has been received. Therefore, a patient can control the inhibition of the normal stimulation process for a predetermined amount of time by analyzing the type of signal that has been sent to the IMD 200. Utilizing embodiments of the present invention, flexibility relating to the normal safety reaction to magnetic signal, or inhibition of normal signal stimulation may be achieved by preprogrammed inputs and/or by the input from the patient and/or the physician.

The particular embodiments disclosed above are illustrative only as the invention may be modified and practiced in different but equivalent manners apparent to those skilled in the art having the benefit of the teachings herein. Furthermore, no limitations are intended to the details of construction or design herein shown other than as described in the claims below. It is, therefore, evident that the particular embodiments disclosed above may be altered or modified and all such variations are considered within the scope and spirit of the invention. Accordingly, the protection sought herein is as set forth in the claims below.

What is claimed:

1. A method for altering a neurostimulation therapy provided by an implantable medical device (IMD), comprising:
providing an implantable medical device capable of providing a neurostimulation therapy comprising at least a first electrical signal;
programming the implantable medical device with a time period for altering the neurostimulation therapy;
detecting the presence of at least one of a magnetic field and a tap input; and
altering said neurostimulation therapy for the duration of said programmed time period, wherein said alteration comprises at least one of inhibiting the first electrical signal and performing a background stimulation.

2. The method of claim 1, wherein detecting the presence of a magnetic field comprises determining at least one of a duration of said magnetic field and a magnitude of said magnetic field.

3. The method of claim 1, wherein detecting the presence of a tap input comprises determining a number of tap inputs received.

4. The method of claim 1, wherein altering said neurostimulation therapy comprises performing a background stimulation, and wherein said background stimulation comprises a reduced stimulation.

5. The method of claim 1, wherein altering said neurostimulation therapy comprises inhibiting said first electrical signal and performing a background stimulation, and wherein said background stimulation comprises a sub-side effect stimulation.

6. The method of claim 1, wherein programming the implantable medical device with a time period comprises programming a time period between zero seconds to about 64,000 seconds.

7. A method for altering a neurostimulation therapy provided by an implantable medical device (IMD), comprising:
programmably defining a time period for altering a neurostimulation therapy;
receiving an input from a source external to said IMD; and
inhibiting said neurostimulation therapy for the programmably defined time period in response to receiving said input.

8. The method of claim 7, wherein receiving said input from said external source comprises receiving at least one of a tap input and a magnetic input.

9. The method of claim 7, further comprising programmably defining a plurality of time periods for altering said neurostimulation therapy, and wherein inhibiting said neurostimulation therapy comprises selecting one of said plurality of time periods for altering said neurostimulation therapy based upon a characteristic of said input, and inhibiting said neurostimulation therapy comprises inhibiting said neurostimulation therapy for said selected time period.

10. The method of claim 9, wherein receiving an input comprises receiving a magnetic field and determining at least one of a duration of said magnetic field and a magnitude of said magnetic field, and wherein selecting one of said plurality of time periods comprises selecting one of said time periods based upon one of a duration of said magnetic field and a magnitude of said magnetic field.

11. The method of claim 9, wherein receiving an input comprises receiving a tap input and determining a number of tap inputs received, and wherein selecting one of said plurality of time periods comprises selecting one of said time periods based upon the number of tap inputs received.

12. The method of claim 7, wherein programmably defining a time period comprises defining a time period of a duration of an integer value between zero seconds and about 64,000 seconds.

13. A method for altering a neurostimulation therapy provided by an implantable medical device (IMD), comprising:
providing an implantable medical device capable of providing a neurostimulation therapy comprising a first electrical signal;
determining at least one programmed alteration time period exceeding 60 seconds;
detecting the presence of at least one of a magnetic field and a tap input; and
altering said neurostimulation therapy for said programmed alteration time period, wherein said alteration comprises at least one of inhibiting the first electrical signal, performing a background stimulation, performing a reduced stimulation, performing a sub-side effect stimulation, and performing an imperceptible stimulation.

14. The method of claim 13, wherein determining at least one programmed alteration time period comprises determining a plurality of alteration time periods, and wherein altering said neurostimulation therapy for said alteration time period comprises selecting one of said plurality of alteration time periods based upon a characteristic of said input.

15. The method of claim 13, wherein said altering comprises inhibiting the first electrical signal and performing a reduced stimulation.

16. The method of claim 15, wherein the reduced stimulation comprises a sub-side effect stimulation.

17. The method of claim 15, wherein the reduced stimulation comprises an imperceptible stimulation.

18. The method of claim 13, wherein said altering comprises performing a background stimulation.
